(19) United States
(12) Patent Application Publication
(10) Pub. No.: US 2007/0270707 A1
(43) Pub. Date: Nov. 22, 2007

(54) MONITORING FLUID IN A SUBJECT USING A WEIGHT SCALE

(76) Inventor: Andres Belalcazar, St. Paul, MN (US)

Correspondence Address: SCHWEGMAN, LUNDBERG & WOESSNER, P.A., P.O. BOX 2938, MINNEAPOLIS, MN, 55402, US

(21) Appl. No.: 11/419,120

(22) Filed: May 18, 2006

(51) Int. Cl. A61B 5/05

(52) U.S. Cl. 600/547

(57) ABSTRACT

Changes in an amount of fluid in a region of a subject, such as the lung(s), may be detected by internally injecting a current through the region, detecting a resulting voltage at an upper and lower body portion, and calculating an impedance value using knowledge of the injected current and resulting voltage. Alternatively, the amount of fluid in the region may be found by internally applying a voltage of known or controllable value (thereby, injecting a current), detecting a resulting voltage at the upper and lower body portions, and calculating a fluid indicative signal using the resulting voltage or using a product of the resulting voltage and the injected current (i.e., power). A method for performing such measurements includes, among other things, injecting a current between first and second internal electrodes and measuring a resulting voltage between first and second external electrodes contacting the subject's upper and lower body portions.

### TECHNICAL FIELD

This patent document pertains generally to measuring an amount of fluid in an internal organ, such as a lung. More particularly, but not by way of limitation, this patent document pertains to monitoring fluid in a region of a subject using, at least in part, a weight scale device and methods related thereto.

### BACKGROUND

Variations in how much fluid is present in a subject's thoracic region can take various forms and can have different causes. As one example, eating salty foods can result in the retainment of excessive fluid in the thorax, which is commonly referred to as “thoracic fluid,” and elsewhere. Another source of fluid build-up in the thorax is pulmonary edema, which involves a build-up of extravascular fluid in or around the lungs.

One cause of pulmonary edema is congestive heart failure (referred to as “CHF”), which is also sometimes referred to as “chronic heart failure,” or simply as “heart failure.” CHF may be conceptualized as an enlarged weakened heart muscle. The impaired heart muscle results in poor cardiac output of blood. As a result of such poor blood circulation, blood tends to pool in blood vessels in the lungs and becomes a barrier to normal oxygen exchange. In brief, pulmonary edema may be an indicative and important condition associated with CHF.

Pulmonary edema, if it exists, may present a medical emergency that requires immediate care. While it can sometimes prove fatal, the outlook for subjects possessing pulmonary edema can be good upon early detection and prompt treatment of the same. If left undetected (and consequently untreated), pulmonary edema may lead to death.

It is possible to detect fluid in the thoracic region by making one or more electrical impedance measurements across the lungs (such measurements commonly referred to as “thoracic impedance”). The more fluid that is present in the lungs, the lower the electrical impedance that results. One way electrical impedance may be measured is by using an implantable medical device (referred to as “IMD”), such as a pacemaker or defibrillator, implanted in a chest area of a subject. Typically, an electrical impedance measurement is made between one or more right ventricular chamber electrodes connected to the implanted device (e.g., via a lead), and another electrode at the implanted device itself In this way, the impedance measurement may sample thoracic tissues, including the lungs.

### BRIEF DESCRIPTION OF THE DRAWINGS

In the drawings, which are not necessarily drawn to scale, like numerals describe substantially similar components throughout the several views. The drawings illustrate generally, by way of example, but not by way of limitation, various embodiments discussed in the present document.

FIG. 1 is a block diagram illustrating exemplary causes and indications of abnormal fluid build-up in a subject's lungs, such as may be the result of pulmonary edema.

FIG. 2A is a perspective view illustrating an electrode configuration providing a positive sensitivity region within or near a subject's lung.

FIG. 2B is a perspective view illustrating an electrode configuration providing a negative sensitivity region within or near a subject's lung.

FIG. 3 is a schematic view illustrating an exemplary system adapted to monitor thoracic fluid in a subject, including an IMD, a weight scale device, a home station device including a processing unit, a communication network, and one or more data storage.

FIG. 4A is a perspective view illustrating portions of an exemplary system adapted to monitor thoracic fluid in a subject and a lead field associated with one or more electrodes of such system.

FIG. 4B is a perspective view illustrating portions of an exemplary system adapted to monitor thoracic fluid in a subject and a lead field associated with one or more electrodes of such system.

FIG. 5A is a perspective view illustrating portions of an exemplary system adapted to monitor thoracic fluid in a subject and lead field junctions associated with electrodes of such system.

FIG. 5B is a perspective view illustrating portions of an exemplary system adapted to monitor thoracic fluid in a subject and lead field junctions associated with electrodes of such system.

FIG. 6 is a bar chart from a computer simulation illustrating enhanced monitoring of thoracic fluid in a subject made possible by the present systems, devices, and methods.

FIG. 7 is a flow chart illustrating an exemplary method providing monitoring of fluid in a region of a subject.

FIG. 8A is a graph illustrating a trend in calculated thoracic impedance that may indicate an increased fluid build-up in a subject's lungs or other region of interest.

FIG. 8B is a graph illustrating a trend in sensed voltage resulting from an internally injected stimulus that may indicate an increased fluid build-up in a subject's lungs or other region of interest.

### DETAILED DESCRIPTION

The following detailed description includes references to the accompanying drawings, which form a part of the detailed description. The drawings show, by way of illustration, specific embodiments in which the present systems, devices, and methods may be practiced. These embodiments, which are also referred to herein as “examples,” are described in enough detail to enable those skilled in the art to practice the present systems, devices, and methods. The embodiments may be combined, other embodiments may be utilized or structural, electrical, or logical changes may be made without departing from the scope of the present systems, devices, and methods. The following detailed description is, therefore, not to be taken in a limiting sense, and the scope of the present systems, devices, and methods are defined by the appended claims and their legal equivalents.

In this document, the terms “a” or “an” are used to include one or more than one; the term “or” is used to refer to a nonexclusive “or” unless otherwise indicated; the term “subject” is used to include the term “patient”; and the term “thorax” refers generally to a subject's body between the neck and the diaphragm. In addition, it is to be understood that the phraseology or terminology employed herein, and not otherwise defined, is for the purpose of description only and not of limitation.

Furthermore, in the event of inconsistent usages between this document and those documents so incorporated by reference, the usage in the incorporated references should be considered supplementary to that of this document; for irreconcilable inconsistencies, the usage in this document controls.

Introduction

In general, edema (i.e., an excess fluid buildup in a region of a subject) is a failure or decompensation of one or more homeostatic processes within a subject's body. The body normally prevents the build-up of fluids therewithin by maintaining adequate pressures and concentrations of salt and proteins, and by actively removing excess fluid. If a disease affects any of these normal bodily mechanisms or if the normal bodily mechanisms are unable to keep up with the fluid build-up, the result may be edema, such as pulmonary edema.

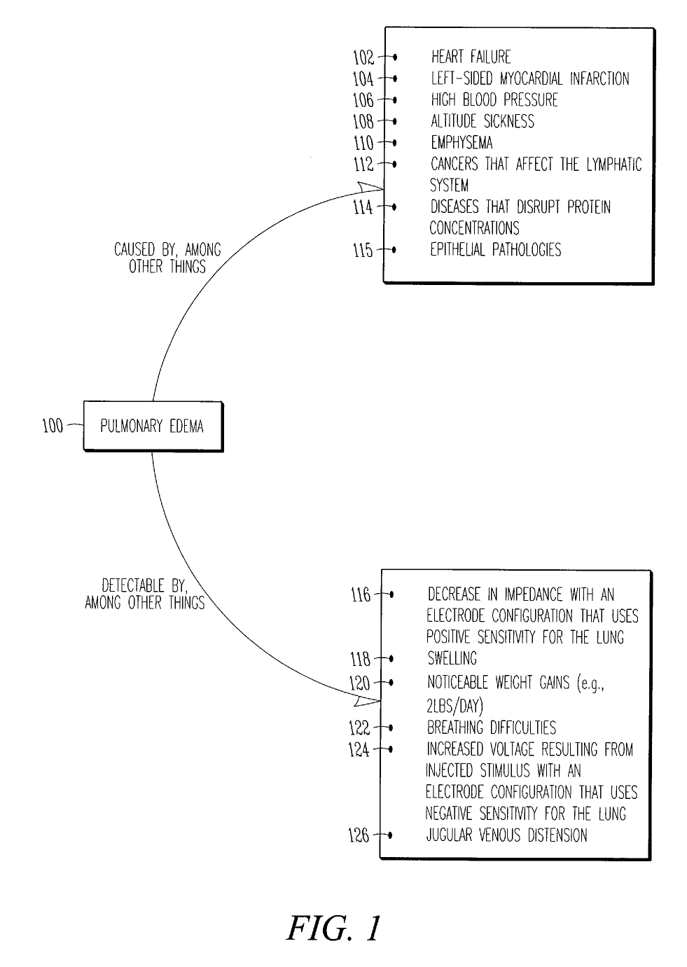

There are several conditions or diseases that may cause or affect pulmonary edema. As shown in FIG. 1, this includes, among others, heart failure 102, left-sided myocardial infarction 104, high blood pressure 106, altitude sickness 108, emphysema 110, cancers that affect the lymphatic system 112, diseases that disrupt protein concentrations 114, or epithelial pathologies 115, such as those caused by inhalation of toxic chemicals, leading to flooding of the alveoli. While pulmonary edema 100 may be a sign of many conditions or diseases, the prospect that pulmonary edema 100 may be a sign of failing heart circulation 102 is often of first concern to caregivers (e.g., physicians) due to the severity of its nature.

Unfortunately, the first indication that an attending caregiver typically has of an occurrence of pulmonary edema 100 is very late in the disease process, such as when it becomes a physical manifestation with swelling 118, noticeable weight gains 120, jugular venous distension 126, or breathing difficulties 122 so overwhelming as to be noticed by the subject who then proceeds to be examined by his/her caregiver. For a heart failure subject, hospitalization at such a (physically apparent) time would likely be required.

Today, heart failure is a major cause of hospital admissions. A portion of these admissions is due to fluid accumulation in the lungs as a result of pulmonary edema 100, which is challenging to treat and often goes unrecognized until a subject is critically ill. It is not unusual for subjects with heart failure to require hospitalization or urgent treatment at an emergency room or critical care unit. It is estimated that approximately 30-40% of subjects with heart failure are hospitalized every year. Further, heart failure is a leading diagnosis-related group among hospitalized subjects over the age of 65.

Morbidity and mortality of heart failure can potentially be lowered with timely detection and appropriate treatment of disease conditions in their early stages, such as upon early detection and treatment of pulmonary edema 100. Early detection and treatment of pulmonary edema 100 may reduce or eliminate the need for hospital admission of subjects with heart failure. A reduction or elimination of the need for hospitalization results in lower health care costs. It is currently estimated that overall expenditures for management and treatment of heart failure may be as high as 24 billion dollars or more per year.

In an effort to detect impending edema and avoid its associated hospitalizations, the present systems, devices, and methods utilize a weight scale in conjunction with concepts of lead field theory. In brief, a lead field can be used to describe a current density vector field that results when a unit of current is injected between at least two electrodes. “Lead field” is a concept that applies to the electrodes injecting current (“current lead field”), as well as to those electrodes measuring resulting voltage (“voltage lead field”). Although a lead field associated with the voltage measurement electrodes may seem surprising at first, as voltage measurement does not entail the injection of current and therefore the creation of an associated lead field in the body, it is sometimes convenient to theoretically conceptualize a current density field resulting from energizing the voltage measurement electrodes with a unit of current.

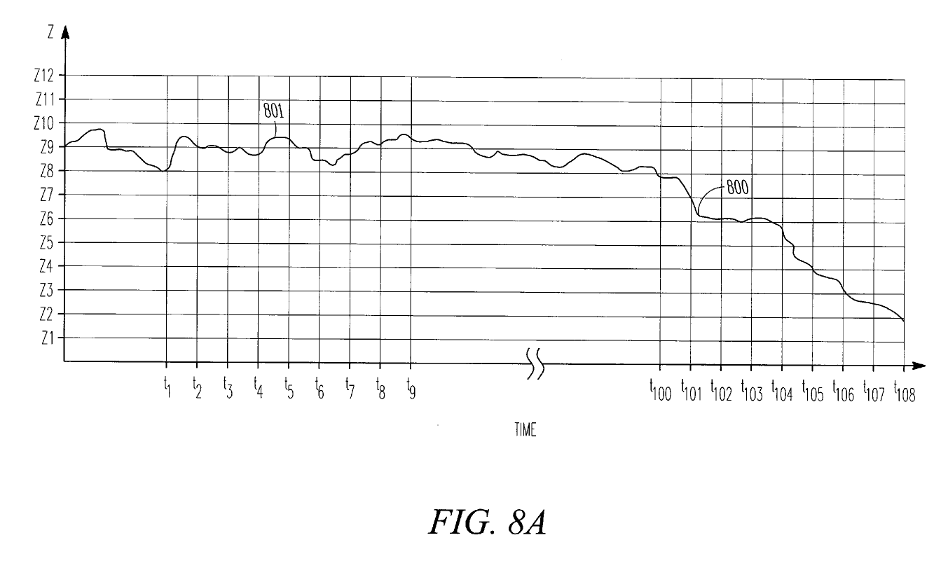

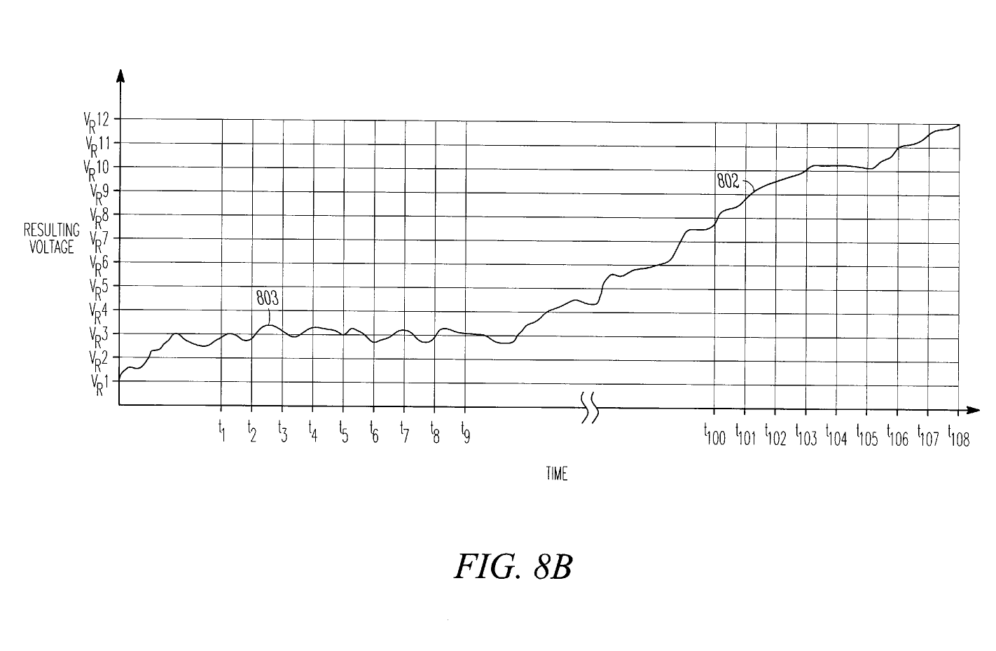

In designing electric systems that monitor fluid amounts within a subject via tissue resistivity changes, it is useful to arrange electrodes in the body so that the current and voltage lead fields intersect at a targeted region with desired geometries and orientations. This allows for high sensitivity in a particular organ (e.g., the lung) or simplification of the circuitry of a monitoring system, thereby potentially reducing its cost. It is possible to arrange the electrodes to create regions within the subject that have positive sensitivity. In a positive sensitivity region, an increase in fluid amount results in a corresponding decrease in the monitored voltage and impedance (see, e.g., FIG. 8A). It is also possible to create regions within the subject that have negative sensitivity, in which an increase in fluid amount results in an increase in the monitored voltage and impedance (see, e.g., FIG. 8B).

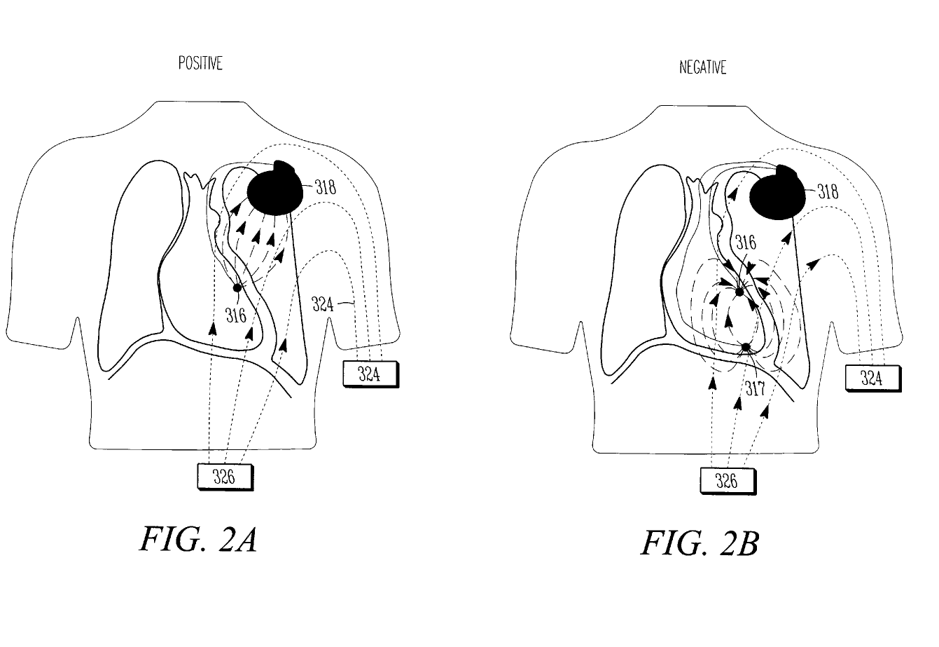

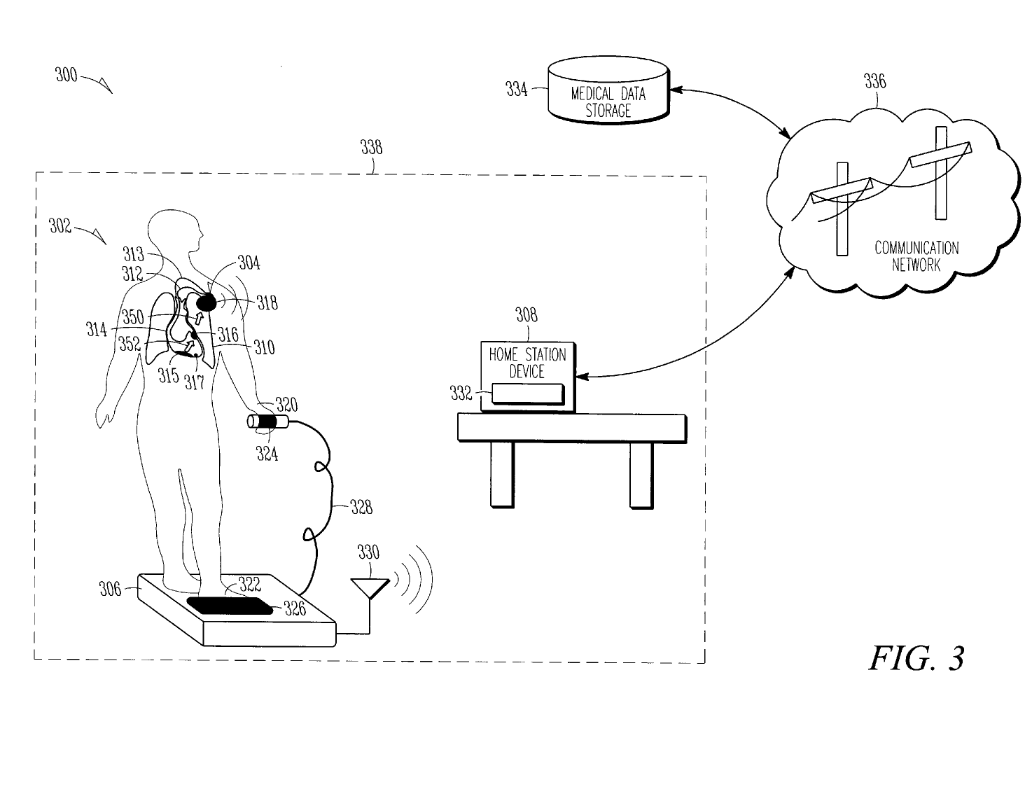

The negativity or positivity of sensitivity in the monitored region is a characteristic of the dot product of the current and voltage lead fields at the desired region's location. For example, in a four electrode system with two electrodes injecting a test current and two other electrodes measuring a resulting voltage, if the current and voltage lead fields have opposing directions (e.g., an angle between the lead field lines is greater than 90 degrees) at the region of interest, such region will be a negative sensitivity region. FIG. 2B illustrates an exemplary electrode configuration giving rise to negative sensitivity monitoring of a subject's lung. On the other hand, if the fields are parallel or substantially parallel (e.g., an angle between the lead field lines is less than 90 degrees), then the region will have positive sensitivity. FIG. 2A illustrates an exemplary electrode configuration giving rise to positive sensitivity monitoring of the subject's lung. As shown in FIG. 2A, the electrodes associated with current injection (e.g., 316, 318) and resulting voltage monitoring (e.g., 324, 326 (FIG. 3)) may both be disposed across the (left) lung to provide substantially parallel lead fields and thus, a positive sensitivity arrangement. As shown in FIG. 2B, the electrodes associated with current injection (e.g., 316, 317) may be disposed outside the lung, while the electrodes associated with resulting voltage monitoring (e.g., 324, 326 (FIG. 3)) may be disposed across the lung to provide opposing lead fields and thus, a negative sensitivity arrangement.

With the above discussed lead theory in mind, the present systems, devices, and methods may advantageously provide enhanced detection of pulmonary edema 100 (FIG. 1) or other abnormal fluid build-up, such as by providing increased sensitivity or by providing more simple monitoring, which may be less costly to implement. This may provide a timelier or cheaper indication of heart failure. As one example, increased fluid build-up within a subject may be monitored with increased sensitivity by monitoring a decrease in thoracic impedance (FIG. 1) when the electrodes are arranged such that the lung is substantially located in a positive sensitivity region. As another example, fluid within a subject may be monitored in a simpler manner by monitoring an increase in a measured voltage resulting from an internally injected stimulus 124 (FIG. 1) when electrodes are arranged such that the lung or other region of interest is substantially located in a negative sensitivity region.

### EXAMPLES

Positive Sensitivity

As discussed above, detection of thoracic fluid (and thus possibly heart failure) may be made by monitoring an impedance of a subject's thoracic region, such as the subject's lungs, when electrodes are arranged to create a positive sensitivity region therein. In this way, a reduction in thoracic impedance 116 (FIG. 1) indicates the presence of an increase in fluid within the lungs. Conversely, a fluid decrease in the lungs corresponds to an increase in thoracic impedance sensed. FIG. 8A illustrates a general decrease 800 in thoracic impedance (Z) as time progresses, such as from time period t₁₀₀-t₁₀₈, and thereby indicates an increase in fluid in the thoracic cavity during such period, which may be the result of pulmonary edema 100. Initially, such as from time period t₀-t₉, FIG. 8A illustrates a substantially stable fluid balance condition as the thoracic impedance trends horizontally 801.

One exemplary technique used for measuring thoracic impedance includes a completely implanted system. On such system includes an IMD to make an electrical impedance measurement between an electrode positioned near a heart and another electrode on the device itself. The IMD is configured to inject an electrical stimulus current of known or attainable value to the one or more implanted electrodes and measure a resulting voltage using one or more other implanted electrodes. Using information about the current and the resulting voltage, the IMD calculates an impedance by taking a ratio of resulting voltage to injected current. This measurement may be repeated over time to detect changes in impedance (and thus changes in fluid amount in the lungs).

Unlike conventionally, wholly implanted impedance systems, the present systems, devices, and methods utilize both internal and external components, and thereby may provide enhanced monitoring of thoracic impedance by, among other things, internally injecting a current through the thoracic region using an IMD, detecting a resulting voltage at an upper body portion (e.g., a hand or shoulder) and a lower body portion (e.g., a foot or lower abdomen) of a subject using an external weight scale device, and calculating an impedance value using information about the injected current and the resulting voltage. As the present subject matter includes both internal (e.g., the IMD) and external (e.g., the weight scale device) components, such approach may be referred to as a “hybrid” approach.

General Discussion

Turning now to FIG. 3, which illustrates one or more internal organs of a subject 302 and a fluid monitoring system 300 including, among other things, an IMD 304, an external weight scale device 306, and a home station device 308 including a processing unit 332. In varying (positive sensitivity) examples, IMD 304, weight scale device 306, and home station device 308 cooperate to measure an electrical impedance of an internal organ or region, such as a left lung 310. In varying (negative sensitivity) examples, IMD 304, weight scale device 306, and home station device 308 cooperate to monitor fluid status of an internal organ or other region of interest through simple monitoring of voltage (resulting from an injected current) or using a product of the resulting voltage and injected current (i.e., detect edema using power), both of which do not require a calculation of electrical impedance.

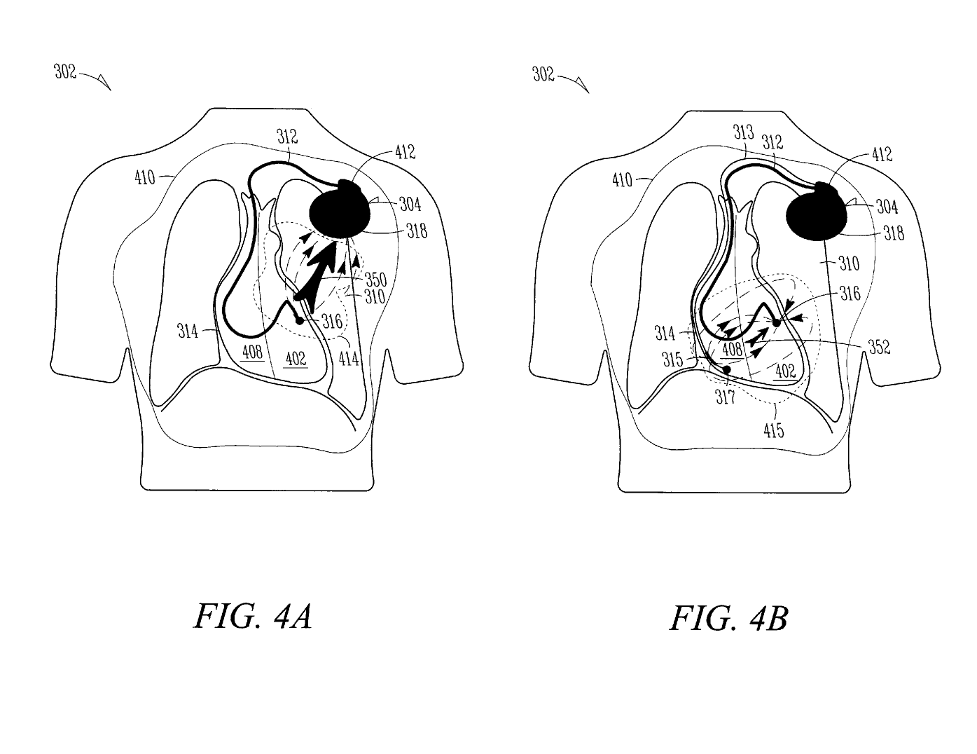

In this example, IMD 304 is implanted subcutaneously in the subject's chest and is designed to inject an electrical current 350 or 352 into subject 302, and in some examples, may further detect or treat irregular cardiac conditions, via one or more electrodes, such as on electrically coupled implantable leads 312, 313. As shown in this example, a first lead 312 has, near its intermediate portion or distal end, an electrode 316 position within, over, or about a left ventricle 402 (FIG. 4A) of a subject's heart 314; while a second lead 313 has near its intermediate portion a coil electrode 315 and near its distal end a tip electrode 317, both of which are positioned within, over, or about a right ventricle 408 (FIG. 4A) of heart 314.

As used herein, IMD 304 may include, but is not limited to, cardiac rhythm management (referred to as “CRM”) devices such as pacemakers, cardioverters, defibrillators; cardiac resynchronization therapy (referred to as “CRT”) or coordination devices, drug delivery systems, or any other device or combination of devices adapted to deliver an electrical stimulation pulse (e.g., a pacing pulse). The one or more leads 312, 313 typically include at least one electrode (e.g., 315, 316, or 317). In some IMDs 304, a housing 318 of the IMD is conductive and serves as a “can” electrode.

Positive Sensitivity

According to one exemplary fluid monitoring operation (using, for example, the system 300 shown in FIG. 3), IMD 304 injects an electrical current 350 that flows from internal electrode 316 (i.e., the electrode within, over, or about left ventricle 402) through at least a portion of left lung 310 to the IMD housing 318, which serves as an internal can electrode. Injected electrical current 350 results in a voltage signal, among other places, at an upper 320 and a lower 322 limb of subject 302. To sense the resulting voltage, a first external electrode 324 is positioned to contact upper limb 320, such as a subject's hand, and a second external electrode 326 is positioned to be in contact with lower limb 322, such as a subject's foot. In this example, first external electrode 324 comprises a handle electrode and second external electrode 326 is disposed on a top surface of weight scale device 306 such that it becomes contacted when subject 302 steps onto the scale. The resulting voltage signal relates to the fluid status of the internal organ, such as left lung 310, to be measured. In one example, the injected electrical current 350 may be accomplished (i.e., generated) by a constant current source. In another example, injected electrical current 350 may be accomplished by applying a pacing voltage between internal electrodes 316, 318 using IMD 304.

Negative Sensitivity

According to another exemplary fluid monitoring operation (using, for example, the system 300 shown in FIG. 3), IMD 304 injects an electrical current 352 that flows from internal electrode 316 to a right ventricular electrode 315 or 317, on second lead 313. Injected electrical current 352 results in a voltage signal, among other places, at upper 320 and lower 322 body portions of subject 302. To sense the resulting voltage, a first external electrode 324 is positioned to contact an upper limb 320, such as the subject's hand, and a second external electrode 326 is positioned to contact a lower limb 322, such as the subject's foot. In one example, the injected current 352 may be accomplished (i.e., generated) by applying a constant voltage source (as provided by, for example, a leading edge of a pacing pulse) between internal electrodes 316 and 315 or 317 using IMD 304. The resulting voltage signal relates to the fluid status of the internal organ, such as left lung 310, to be measured. In this example, even though a direct line between electrodes 316 and 315 or 317 does not intersect left lung 310, a good sensitivity to left lung fluid changes is achieved by way of a lead field 415 (FIG. 4B) that radiates from electrode 316, which may cause significant currents to flow through the left lung.

General Discussion

Referring still to FIG. 3, a cable 328 electrically couples first external electrode 324 and second external electrode 326 allowing for a value of the resulting voltage signal to be determined. In one example, weight scale device 306 is adapted to communicate the resulting voltage signal (wirelessly via telemetry using an antenna 330 and associated telemetry circuitry or via a second cable (not shown) connected) to a processing unit 332 (which in this example is, but need not be) integral with home station device 308. While processing unit 332 and home station device 308 are illustrated as being elements distinct from weight scale device 306 and IMD 304, the present systems, devices, and methods are not so limited. In another example, one or both of processing unit 332 or home station device 308 are incorporated into weight scale device 306 or IMD 304.

In one example, IMD 304 includes circuitry adapted to measure a value of injected current 350 or 352 directly. Alternatively, injected current 350 or 352 may be of a known value, as provided by, for example, a constant current source. In another example, IMD 304 is adapted to measure one or more parameters that allow for the value of injected current 350 or 352 to be known or determined, such parameters including an (IMD 304) applied pacing voltage and a lead impedance (i.e., an impedance into which IMD 304 injects current 350 or 352, which typically includes the impedance of bodily tissue as well as that of lead 312 or 313 itself—notably, lead impedance is generally approximated by the impedance of lead 312 or 313 only). In all such examples, information about injected current 350 or 352 may be communicated (e.g., wirelessly via telemetry using an internal antenna) to processing unit 332. The telemetry may employ various wireless techniques such as infrared, ultrasound, magnetic fields, radio frequency (referred to as “RF”), etc.

Positive Sensitivity

Upon receiving information about the resulting voltage signal from weight scale device 306 and information about the injected current 350 from IMD 304, processing unit 332 is adapted to compute a value of injected current 350 (if necessary), and divide a value of the resulting voltage by the value of the injected current 350 thereby determining an impedance characteristic of, for example, left lung 310. Repeating the foregoing measurements and computations over a period of time, and with the same (or similar) configurations and positioning of the internal and external electrodes may yield a monitorable change of organ fluid status, such as left lung 310 fluid status. Successive organ impedance values may be compared with one another to detect changes in impedance values that may correspond to changes in fluid accumulation within the associated organ. In this example, the fluid status of left lung 310 may be measured a number of times, with the impedance values thereafter compared to detect changes in fluid status.

Because internal organs, such as left lung 310, have electrical resistance, electric field laws predict that a flow of current (e.g., injected current 350) will resulting in a voltage across organs in subject 302, as well as on the surface of the subject's body (e.g., at one or more limbs 320, 322). As fluid content in the organ increases, the resistivity of the organ decreases, and, for a given current, the resulting voltage at the upper and lower limbs 320, 322, respectively, also decreases. The thoracic impedance (Z) (FIG. 8A), which (as discussed above) may be computed by dividing the resulting voltage between upper 320 and lower 322 limbs, by injected current 350 can be determined from Ohm's law (i.e., Z=resulting voltage/injected current).

Processing unit 332 or home station device 308 may store the calculated thoracic impedance values in a memory for later recall, purposes of trending, displaying one or more impedance results to an operator, or transmitting the results to a remote health care provider or data storage 334 using a communication network 336, such as an Internet or a telephone connection. In brief, home station device 308 or processing unit 332 may perform one or more of the following functions: gathering data (e.g., data related to injected current 350 or 352 or the resulting voltage), calculating one or more thoracic impedance values, sending the data or thoracic impedance values to a remote health care provider or medical data storage 334, or receiving one or more commands from the remote health care provider for transmission to IMD 304 or weight scale device 306.

Nezative Sensitivity

While portions of the foregoing discusses monitoring fluid status within a region (e.g., left lung 310) of a subject 302 using calculated impedance, the present subject matter is not so limited. Indications of fluid within a region of subject 302 may also be determined using weight scale 306 (or other external device) without the need for impedance calculations and information needed to make such calculations, as is described in commonly assigned Belalcazar, U.S. patent application Ser. No. ______, entitled “MONITORING FLUID IN A SUBJECT USING AN ELECTRODE CONFIGURATION PROVIDING NEGATIVE SENSITIVITY REGIONS,” filed even date herewith (Attorney Docket No. 279.C39US1), which is hereby incorporated by reference in its entirety. Among other things, U.S. Patent Application, entitled “MONITORING FLUID IN A SUBJECT USING AN ELECTRODE CONFIGURATION PROVIDING NEGATIVE SENSITIVITY REGIONS” discusses systems and methods utilizing an electrode configuration providing a negative sensitivity region (see, e.g., FIG. 2B) to monitor fluid changes in an organ, such as a lung. It has been found that by using a negative sensitivity electrode configuration, monitoring of fluid levels within subject 302 may be performed by using a constant voltage source (as given, for example, by the leading edge of a pacing pulse), and through simple monitoring of sensed resulting voltage without the need to calculate an impedance or provide a constant current source. The resulting voltage may be measured by external electrodes 324, 326 associated with weight scale 306.

Alternatively, fluid monitoring in the organ of interest may be monitored with the same system just described, using a product of the resulting voltage and the injected current 352. In this case, the monitored quantity may be thought of as a partial measure of dissipated power in, for example, the thorax, since power dissipated by a resistive load is the product of the current flowing through it times the voltage it has. In the example of left lung 310 located in a negative sensitivity arrangement, the more edema fluid the lung has, the more the voltage in the limbs (for example) increases, such that the power available and measured elsewhere in the body will consequently be increased as well. This increase in power appearing the body can be monitored using, in part, weight scale 306, which contacts upper 320 and lower 322 body portions to measure the power appearing in a substantial portion of the subject's thorax. Using the power to monitor fluid status takes advantage of the synergistic increases in injected current and resultant voltage that occur when edema fluid appears in a targeted organ. The multiplication of these two synergistic quantities amplifies the measurement signal of the developing edema, yielding a more sensitive system to the fluid in the targeted organ.

General Discussion

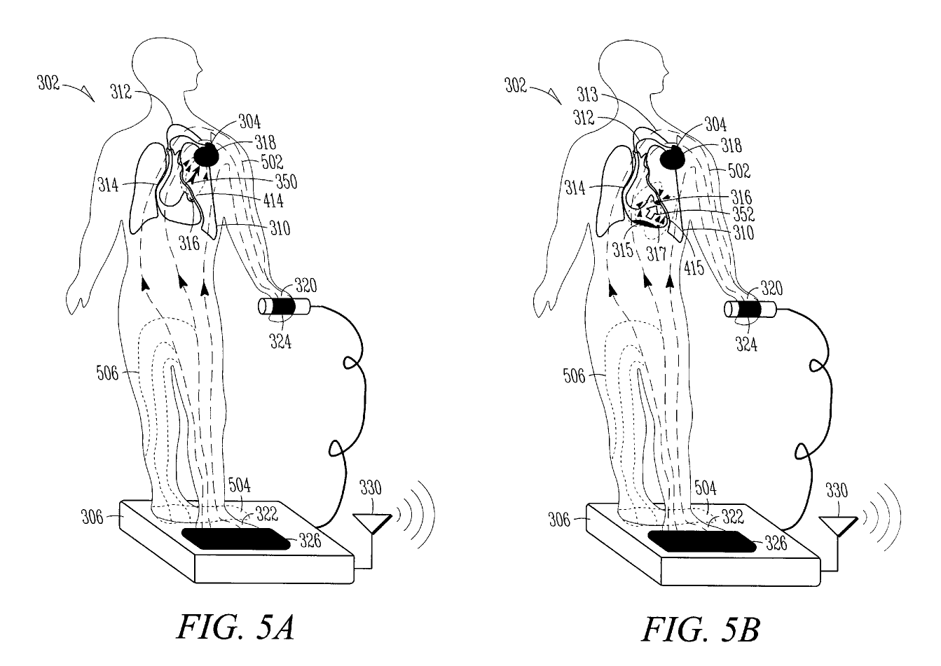

In addition to detecting the resulting voltage, weight scale device 306 may be adapted to provide many other types of information to processing unit 332 or home station device 308, all of which may be helpful in determining a fluid status within subject 302. As one example, weight scale device 306 may be adapted to measure a weight of subject 302 and transmit a signal indicative of a subject's weight to processing unit 332 or home station device 308. An increase in weight (e.g., 2 or more lbs./day) may correlate to an indication of abnormal fluid build-up, such as that accompanying pulmonary edema. As another example, weight scale device 306 may be adapted to detect one or more impedance signals indicative of lower limb (e.g., ankle) edema (see, e.g., FIG. 5). A presence of ankle edema may correlate to an indication of abnormal fluid build-up, such as that accompanying pulmonary edema.

Weight scales are part of an established practice of using home-based 338 medical devices intended to manage heart failure subjects. Advantageously, the present systems, devices, and methods make use of this established practice by incorporating into a weight scale device (e.g., 306), among other things, the above-discussed functions for the monitoring of fluid within a subject 302. Another advantage of the present systems, devices, and methods includes the concept that weight scale device 306 may be adapted to work with any IMD 304, even those that don't have associated thoracic edema capabilities.

FIGS. 4A-4B illustrate portions of a fluid monitoring system 300 (FIG. 3), such as an IMD 304 and one or more electrode bearing leads 312, 313, and lead fields 414, 415 associated therewith. In each FIG., a section of subject 302 is shown with a cut-away area 410 to allow for illustration of IMD 304 and the electrode bearing leads 312, 313, among other things.

Positive Sensitivity

In the example of FIG. 4A, IMD 304 has two internal electrodes 316, 318 associated with energizing of such electrodes. A first internal electrode 316 is disposed on an intermediate or a distal portion of a first lead 312, which is coupled with IMD 304. A housing 318 of the IMD acts as a second internal (can) electrode by being conductive or at least partially conductive. Lead 312 extends between electrodes 316 and 318 and thereby provides a conductive path from ND 304 to electrode 316. In this way, when IMD 304 provides an electrical stimulus (e.g., a constant current source or an applied pacing pulse), lead 312 and electrode 316 deliver the stimulus through one or more internal organs as an injected current 350, the latter of which returns to the IMD by way of conductive housing 318. As shown, a lead field 414 is associated with current injection 350.

Negative Sensitivity

In the example of FIG. 4B, IMD 304 has at least three internal electrodes 315, 316, 317 associated therewith. A first internal electrode 316 is disposed on an intermediate or a distal portion of a first lead 312 coupled with IMD 304. A second 315 and a third 317 internal electrode are disposed on an intermediate or a distal portion of a second lead 313, which is also coupled with IMD 304. Leads 312, 313 extend between electrodes 315, 316, 317, and IMD 304 and thereby provide a conductive path from the IMD to the electrodes. In this way, when IMD 304 provides an electrical stimulus (e.g., a constant voltage source given by, for example, a leading edge of an applied pacing pulse), leads 312, 313 and electrode 315, 316, 317 deliver the stimulus through one or more internal organs as an injected current 352. As shown, a lead field 415 is associated with current injection 352.

General Discussion

In one example, IMD 304 includes a current measurement capability to measure injected current 350 or 352 that flows between the current injection electrodes, and thus through the tissues and organs therebetween. Alternatively, injected current 350 or 352 may be of a known fixed magnitude, as provided by a current source circuit, which injects the same amount of current independent of loading. In another example, IMD 304 includes circuitry to measure lead impedance. This value, along with a programmed or (IMD) measured pacing applied voltage value, allows for the determination of injected current 350 or 352 using Ohm's law.

A desirable position of electrodes 315, 316, 317, and 318 may be determined using several factors. For instance, one factor that determines the position of the electrodes is that a (current injection) electrode pair be positioned such that the organ of interest, such as the left lung 310, receives the maximum available current density associated with injected current 350 or 352 as is possible. In the exemplary electrode configuration shown in FIG. 4A, the intermediate or distal portion of lead 312 is positioned such that electrode 316 is located within, over, or about a left ventricle 402 of a heart 314 such that a significant fraction of injected current 350 flows through a portion of left lung 310. It has been found that an electrode associated with the left ventricle provides unique (high) lung sensitivity.

In alternative examples, the second injection electrode—or return electrode—may be separate from housing 318 of IMD 304 (e.g., on a header 412 of the IMD or located on another lead), thereby defining a different path for injected current. For instance, as shown in FIG. 4B, the second injection electrode could be a right ventricle coil electrode 315 disposed on a second lead 313 (FIG. 3). Any repositioning of an injection electrode in system 300 would change the path taken by current 350 or 352 (FIG. 3) and thereby change the impedance measured. As such, separating the second injection electrode from housing 318 may allow for greater flexibility in targeting an organ for which the impedance is to be measured.

FIGS. 5A-5B illustrate one or more internal organs of a subject 302 and portions of a fluid monitoring system 300 (FIG. 3) including, among other things, an IMD 304 and a weight scale device 306. These FIGS further illustrate lead fields created by a current injection electrode pair and a lead field created by a voltage measurement electrode pair, which intersect around a thoracic region of subject 302. As discussed above, it is possible to arrange the current and voltage electrodes to create regions within the subject that have positive sensitivity, where an increase in fluid amount results in a corresponding decrease in the monitored voltage and impedance (see, e.g., FIG. 8A). It is also possible to create regions within the subject that have negative sensitivity, where an increase in fluid amount results in an increase in the monitored resulting voltage 802 (see, e.g., FIG. 8B, which illustrates a substantially stable fluid balance condition from time period t₀-t₉—as the resulting voltage trends horizontally 803—and a general increase 802 in resulting voltage (V_{R}) as time progresses, such as from time period t₁₀₀-t₁₀₈, and thereby indicates an increase in fluid in the thoracic cavity during such period, which may be the result of pulmonary edema 100). To reiterate, in negative sensitivity regions, fluid monitoring within an organ may be performed by monitoring the resulting voltage, or alternatively, monitoring power (i.e., a product of the injected current and the resulting voltage).

As also discussed above, the negativity or positivity of sensitivity in the monitored region is a characteristic of the dot product of the current and voltage lead fields at the desired region's location. For example, in a four electrode system with two electrodes injecting a test current and two other electrodes measuring a resulting voltage, if the current and voltage lead fields have opposing directions (e.g., an angle between the lead field lines is greater than 90 degrees) at the region of interest, as is shown in FIG. 5B, such region will be a negative sensitivity region.

On the other hand, if the fields are parallel or substantially parallel (e.g., an angle between the lead field lines is less than 90 degrees), as is shown in FIG. 5A, then the region will have positive sensitivity.

Positive Sensitivity

FIG. 5A illustrates a lead field 414 created by current injection electrodes 316, 318 and a lead field 502 created by voltage measurement electrodes 324, 326 that intersect around the subject's left lung 310. A first lead 312 is coupled to IMD 304 and extends from the device to a portion of heart 314, such as a left ventricle 402 (FIG. 4). In this example, first lead 312 includes internal electrode 316 on an intermediate or a distal portion thereof A housing 318 of IMD 304 acts as another internal electrode. Thus, when circuitry associated with ND 304 provides an electrical stimulus (e.g., a constant current source or an applied pacing pulse), an injected current 350 may be delivered between, for example, electrode 316 and electrode 318. IMD 304 may be adapted to wirelessly transmit information about injected current 350 to a processing unit 332 (FIG. 3). As shown, the delivery of injected current 350 creates lead field 414 extending between the injection electrodes 316 and 318.

Injection current 350 results in a voltage being created, among other places, at an upper 320 and a lower 322 limb portion of subject 302. In this example, a value of the resulting voltage signal is sensed using a first external electrode 324 in contact with upper limb 320 (e.g., a hand) and a second external electrode 326 in contact with lower limb 322 (e.g., a foot). Lead field 502 created by the voltage measurement electrodes is shown extending between the subject's hand and foot and represents an electric field that would exist if electrodes 324 and 326 were reciprocally energized (as was, for example, internal electrode 316). First 324 and second 326 external electrodes are electrically coupled to weight scale device 306, which may be adapted to transmit (e.g., using antenna 330) the resulting voltage signal detected by the electrodes to processing unit 332.

From the received resulting voltage and injected current 350 information, processing unit 332 may calculate impedance, thereby providing an indication of organ fluid status. The impedance of a tetrapolar system 300, such as is shown in FIG. 5A, may be theoretically conceptualized as being determined by the intersection of the lead field 414 (created by current injection electrodes 316, 318) and the lead field 502 (created by voltage measurement electrodes 324 and 326, if reciprocally energized). As illustrated, lead field 414 covers portions of left lung 310, and intersects at the left lung 310 with angles less than 90 degrees by (rather uniform) lead field 502 extending through the subject's body between his/her hand and foot. Hence, the area of sensitivity in this exemplary configuration is positive for the left lung 310.

Negative Sensitivity

FIG. 5B illustrates a lead field 415 created by current injection electrodes 315, 316 and a lead field 502 created by voltage measurement electrodes 324, 326 that intersect around the subject's heart 314 and left lung 310. A first lead 312 is coupled to IMD 304 and extends from the device to a portion of heart 314, such as a left ventricle 402 (FIG. 4). In this example, first lead 312 includes internal electrode 316 on an intermediate or a distal portion thereof. Also in this example, a second lead 313 including two internal electrodes 315, 317 extend from IMD 304 to a portion of heart 314, such as a right ventricle. A housing 318 of IMD 304 acts as another internal electrode. Thus, when circuitry associated with IMD 304 provides an electrical stimulus (e.g., a constant voltage source given by, for example, a leading edge of an applied pacing pulse), an injected current 352 may be delivered between, for example, electrode 316 and electrode 315. IMD 304 may be adapted to wirelessly transmit information about injected current 352 to a processing unit 332 (FIG. 3). As shown, the delivery of injected current 352 creates lead field 415 extending between the injection electrodes 315 and 316.

Injection current 352 results in a voltage being created, among other places, at an upper 320 and a lower 322 limb portion of subject 302. In this example, a value of the resulting voltage signal is sensed using a first external electrode 324 in contact with upper limb 320 (e.g., a hand) and a second external electrode 326 in contact with lower limb 322 (e.g., a foot). Lead field 502 created by the voltage measurement electrodes is shown extending between the subject's hand and foot and represents an electric field that would exist if electrodes 324 and 326 were reciprocally energized (as was, for example, internal electrode 316). First 324 and second 326 external electrodes are electrically coupled to weight scale device 306, which may be adapted to transmit (e.g., using antenna 330) the resulting voltage signal detected by the electrodes to processing unit 332.

From the received resulting voltage and applied voltage or injected current 352 information, processing unit 332 may calculate a fluid status indicative signal, thereby providing an indication of organ fluid status. Such fluid monitoring, such as is shown in FIG. 5A, may be theoretically conceptualized as being determined by the intersection of the lead field 415 (created by current injection electrodes 315, 316) and the lead field 502 (created by voltage measurement electrodes 324 and 326, if reciprocally energized). As illustrated, lead field 415 covers portions of heart 314 and left lung 310, and intersects at the heart and lungs with angles greater than 90 degrees by (rather uniform) lead field 502 extending through the subject's body between his/her hand and foot. Hence, the area of sensitivity in this exemplary configuration is negative for the heart 314 and lung 310.

General Discussion

In general, what is sought with electrode positioning is to maximize the so-called “dot product” of the current and voltage density lead fields of the electrodes. This product is highly dependent on the current density magnitude, as well as on the orientation of the vectors of the lead fields at the target organ. The internal electrodes 315, 316, 317, or 318 define a first current density field in the thoracic region. The external voltage electrodes 324, 326 define a second vector field. In placing the electrodes optimally, one may seek to maximize the vector dot product in the organ of interest by maximizing the current density in the target organ and minimizing the angle of intersection between vectors of the current density field and vectors of the voltage measurement field. The current density fields of the injection and voltage measurement electrode pairs depend on the electrode positioning as well as on the internal distribution and properties of tissues.

In one example, weight scale device 306 may be further configured to determine a subject's 302 weight and transmit (signals indicative of) such measured weight to processing unit 332 or home station device 308 (FIG. 3) for use in determining a pulmonary edema indication. In another example, the weight scale device 306 may be further configured to monitor ankle impedance of subject 302. In such an example, weight scale 306 may include a third external electrode 504 in contact with a subject's other foot (i.e., the foot not in contact with external electrode 326. A (lower limb) sinusoidal current may be introduced across electrodes 326 and 504 (thereby forming a lower limb lead field 506). A (lower limb) voltage resulting from the (lower limb) current may then be sensed by electrodes 326 and 504. In another example, ankle edema (or lower limb edema) is performed using four foot electrodes, where injection of current occurs between one pair and the voltage sensing occurs in the other pair. In brief, through the use of weight scale device 306, a triple trend measurement including thoracic edema, ankle edema, or weight may be used to provide enhanced monitoring of fluid status, such as pulmonary edema status, within subject 302.

Positive Sensitivity

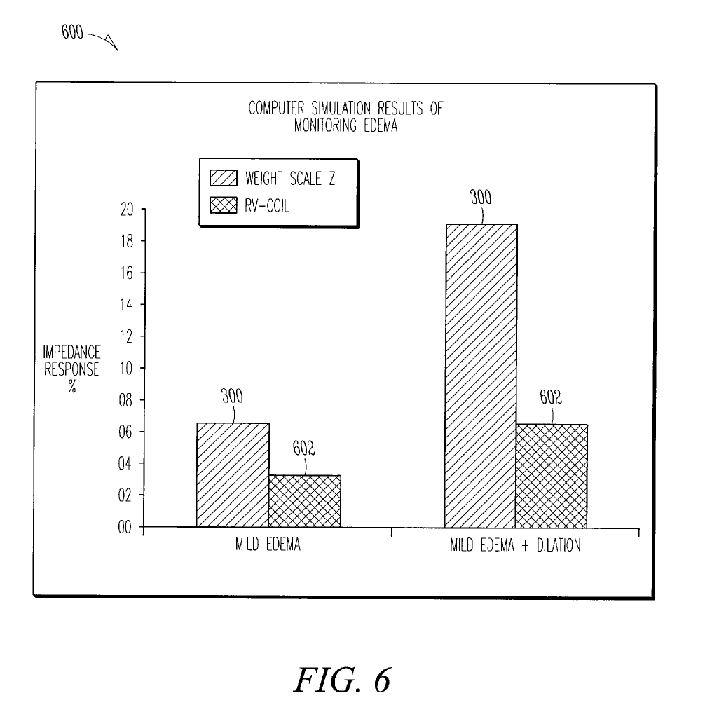

FIG. 6 is a bar chart 600 from a computer simulation comparing the simulated sensitivity of a wholly implanted impedance measuring system with the simulated sensitivity of a hybrid system (e.g., a system 300 (FIG. 3) including both internal (e.g., the IMD 304, leads 312, 313, or electrodes 315, 316, 317, 318) and external (e.g., the weight scale device 306, home station device 308, or electrodes 324, 326) components). The vertical measure of each bar on chart 600 indicates exemplary impedance change levels (as percentages, relative to a healthy baseline of impedance) associated with an episode of pulmonary edema for each configuration determined using computer simulations performed on human thoracic models. Sensitivity can be conceptualized as a measure of the change of impedance resulting from a change in the amount of fluid in an organ, such as a left lung 310 (FIG. 3).

According to at least one computer simulation study, such as is found in Belalcazar, A., Patterson, R., Monitoring lung edema using the pacemaker pulse and skin electrodes, Physiol. Meas. 26 (2005) S153-S163, a wholly implanted impedance system 602 was found to be less sensitive in detecting both mild edema and mild edema in conjunction with heart dilation than a hybrid system, such as a system including skin electrodes. Similar results were found using the hybrid system 300 of the present subject matter, which includes a weight scale. As shown in FIG. 6, hybrid system 300 was found in computer simulation to exhibit a percent increase of approximately 6.5% and 19% in measured impedance for a given change in lung fluid (edema) and lung fluid in conjunction with an enlargement of an organ (dilation), respectively. Wholly implanted system 602 (e.g., right ventricle-coil), on the other hand, was found in computer simulation to exhibit a percent increase of only approximately 3% and 6% for the same given change in lung fluid and lung fluid in conjunction with the enlargement of the organ, respectively. In brief, at least one computer simulation showed that the present hybrid system 300 performs better in regards to impedance sensitivity than a wholly implanted system.

The above-discussed sensitivity analysis was conducted using a computer model. The model simulates lung impedance under normal and edema conditions using a three-dimensional representation that divided a human thorax into many small volumes, each corresponding to body tissue. Each small volume is assigned a resistivity (e.g., blood=150 ohms-cm, normal lung=1400 ohms-cm, muscle=400 ohm-cm, etc.) according to published tables. Electrodes where then placed at an upper and a lower location in the model and current was injected. The computer then calculated the resulting voltage potentials at each of the volumes using electric field equations. The results can be used to compute impedance by dividing the measured potentials by the injected current.

By observing changes in measured impedance that correspond to changes in lung fluid, caregivers may use system 300 to look for trends in impedance indicating that lung fluid is changing over time. Of note, the computed impedance need not be an absolute impedance measure to provide a useful diagnostic tool.

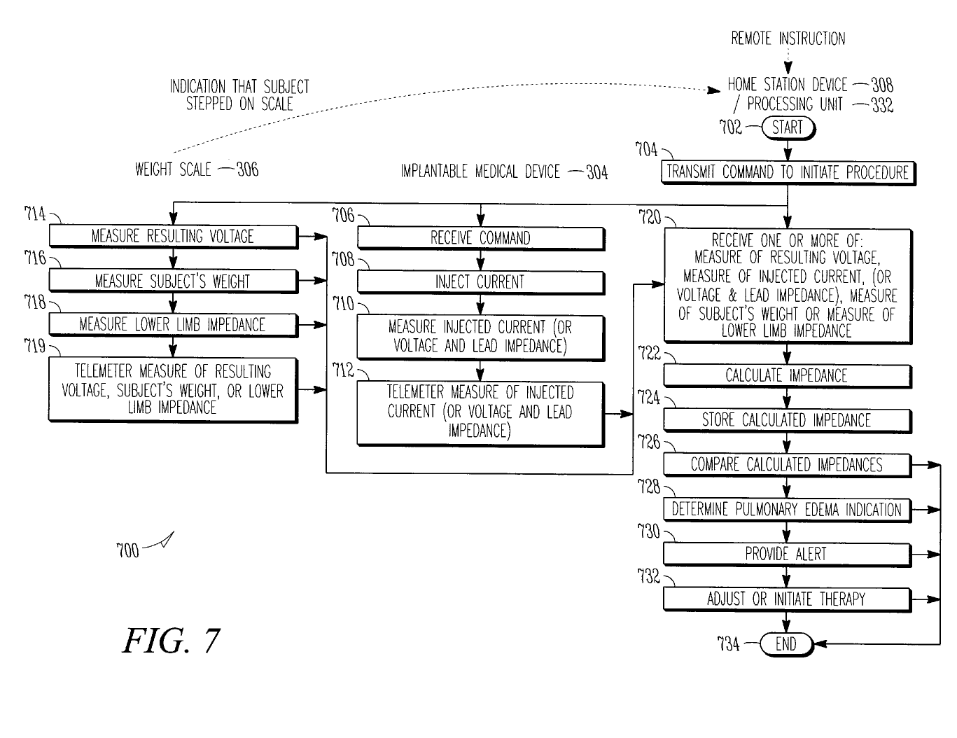

FIG. 7 illustrates a flow chart of an exemplary method 700, which may provide for enhanced monitoring of thoracic fluid in a subject 302 (FIG. 3). In one example, the steps of method 700 may be performed cooperatively using a weight scale device 306, an IMD 304, and a home station device 308 including a processing unit 332. Home station device 308 or processing unit 332, weight scale device 306, and IMD 304 may communicate (e.g., wirelessly via telemetry) to coordinate the receiving of an injected current 350 (FIG. 3) and a resulting voltage by processing unit 332.

In one example, method 700 starts at 702, when a subject initiates the process by stepping onto weight scale device 306 (e.g., process automatically begins when the subject steps on the scale) or when a caregiver (remotely) transmits an instruction to home station device 308 or processing unit 332 to produce an impedance measurement. Upon receiving endorsement from the weight scale device or caregiver, home station device 308 or processing unit 332 transmits a command to IMD 304 to initiate the impedance procedure at 704. At 706, IMD 304 receives the command and proceeds to inject a current 350 (e.g., a constant current pulse of a current generated by applying a pacing voltage), at 708, across a subject's thoracic region. At 710, injected current 350 is measured by ND 304 directly. If IMD is not configured to measure the injected current 350 directly, it may alternatively measure the pacing voltage applied by the IMD (to generate the current) and a lead impedance. Using the pacing voltage and lead impedance, the injected current may be calculated as discussed above. At 712, the measure of injected current 350 or the applied pacing voltage and lead impedance is telemetered to home station device 308 or processing unit 332.

At 714, a resulting voltage is measured by weight scale device 306 using a first electrode in contact with a subject's upper limb (e.g., a subject's hand) and a second electrode in contact with a subject's lower limb (e.g., a subject's foot). Optionally, at 716, a subject's weight may be measured by weight scale device 306, while at 718, a lower limb impedance may optionally be measured. As discussed above, both the subject's weight and information about the presence of lower limb edema (e.g., ankle edema) can help diagnosis a pulmonary edema indication. At 719, the measure of resulting voltage, subject's weight, or lower limb impedance is telemetered to home station device 308 or processing unit 332.

After receiving one or more of the measure of resulting voltage, measure of injected current (or alternatively, pacing voltage and lead impedance), measure of the subject's weight, or measure of the subject's lower limb impedance at 720, the home station device 308 or processing unit 332 calculates, at 722, an impedance using, at least in part, a value of the injected current 350 and the resulting voltage. At 724, the calculated impedance may be stored in a memory in home station device 308 or processing unit 332. At 726, calculated impedances may be compared (to one another) to detect a change in an amount of fluid in the thoracic region. In one example, the calculated impedances may be further compared to a predetermined “specified” threshold value to determine whether a change in fluid amount deserving of attention has occurred.

At 728, a pulmonary edema indication may be determined using the comparison performed at 726 (as discussed, a continuous decrease in measured thoracic impedance may signal a positive pulmonary edema indication). Based on the pulmonary edema indication, an alert may be provided at 730. The alert may be provided in a number of ways. In one example, an audible tone may be sounded, which prompts the subject to call his/her caregiver. If the subject is linked to a remote monitoring system, the alert may also be electronically communicated to the caregiver for review. In another example, the alert is provided to the subject or caregiver at the subject's next office visit. At 732, a therapy is adjusted or initiated in response to the determined pulmonary edema indication. Such therapy may be provided in a number of ways, such as cardiac rhythm management therapy, dietary therapy, or diuretics. In this example, but as may vary, method 700 concludes at or before.

Although FIG. 7 illustrates one exemplary method of monitoring thoracic fluid in a subject, other methods may also be used to perform such monitoring. As one example, IMD 304 may telemeter a lead impedance to home station device 308 or processing unit 332 and a physician may fix a pacing applied voltage, or, if auto-capture is used, the IMD may apply a varying voltage. As such, a value of the pacing applied voltage may be a command (i.e., not a measured) value that is stored in a memory associated with the IMD.

Negative Sensitivity

According to another example, as is discussed and illustrated (see, e.g., FIG. 8) in Belalcazar, U.S. patent application Ser. No. ______, entitled “MONITORING OF FLUID IN A SUBJECT USING AN ELECTRODE CONFIGURATION PROVIDING NEGATIVE SENSITIVITY REGIONS,” filed even date herewith (Attorney Docket No. 279.C39US1), monitoring of thoracic fluid in a subject may be performed via simple monitoring of the measured resulting voltage (i.e., without calculating impedance) when electrodes are arranged such that the thoracic region is substantially located in a negative sensitivity region.

General Discussion

Additional examples included in the present systems, devices, and methods include the following. In one example, IMD 304 (FIG. 3) may not directly measure the injected current signal 350 or 352 (FIG. 3), which can be performed via a series current-sense resistor. Instead, IMD 304 may provide either a known or a measured applied pacing voltage and a measured lead impedance to processing unit 332. In one such example, IMD 304 would telemeter both a value of the applied pacing voltage and a value of the lead impedance to processing unit 332, the latter of which would use the ratio of the applied voltage to the lead impedance as a substitute for a directly measured injected current value.

In another example, implanted (i.e., internal) electrodes 315, 316, 317, 318 may be positioned to inject or receive current in any practical location or position (beyond those discussed above), such as one or more of a right atrium or a left atrium, etc. That is to say, various positions and configurations of electrodes 315, 316, 317, 318 may be used to inject a current or to measure the impedance of internal organs (e.g., left lung 310 (FIG. 3)) in conjunction with the external measurement of resulting voltage caused by the injected current 350 or 352.

While a majority of the foregoing discusses a tetrapolar (i.e., a four-polar) lung impedance determination system (including a two internal electrodes and two external electrodes), the present systems, devices, and methods are not so limited. Additional internal or external electrodes may be used in furtherance, or in lieu, of the electrodes discussed above. Further, internal electrodes may be used that are electrically coupled to the IMD, even if such electrodes are not necessarily implanted to address a cardiac rhythm problem. Further yet, the current injection and voltage measurement electrode pairs discussed herein may be exchanged (i.e., swapped) yielding equivalent measurements (as supported by the Helmholtz theorem of reciprocity). For instance, in one example (as discussed above), an electrode pair associated with an IMD 318 (FIG. 3) is used to inject a current 350 or 352 (FIG. 3) and an electrode pair associated with a weight scale 306 (FIG. 3) is used to measure a resulting voltage; in another example, the electrode pair associated with weight scale 306 is used to inject current 350 or 352 and the electrode pair associated with IMD 318 is used to measure the resulting voltage.

Conclusion

Pulmonary edema is a serious medical condition in which an excessive amount of fluid accumulates in a subject's thoracic region, such as the lungs. This condition may (and oftentimes does) result from heart failure. If it exists, pulmonary edema requires immediate care. While it can sometimes prove fatal, the outlook for subjects possessing pulmonary edema can be good upon early detection and prompt treatment.

Advantageously, the present systems, devices, and methods may provide for enhanced or simplistic monitoring of abnormal fluid amounts in the thoracic region, and thus may provide timely and cost effective detection of thoracic fluid build-up, all in the confines of one's home—without having to make an office appointment or traveling thereto. Such detection is made possible by, among other things, internally injecting a current through the thoracic region (using an IMD), detecting a resulting voltage at an upper and a lower limb of a subject (using a weight scale device), and calculating an impedance value using information about the injected current and the resulting voltage when electrodes are arranged such that the thoracic region is substantially located in a positive sensitivity region. Alternatively, simple fluid monitoring may be performed using the measured resulting voltage without having to further calculate impedances.

It is to be understood that the above description is intended to be illustrative, and not restrictive. For example, the above-described embodiments (or aspects thereof) may be used in combination with each other. Many other embodiments will be apparent to those of skill in the art upon reviewing the above description. The scope of the present systems, devices, and methods should, therefore, be determined with reference to the appended claims, along with the full scope of legal equivalents to which such claims are entitled. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The Abstract of the Disclosure is provided to comply with 37 C.F.R. §1.72(b), requiring an abstract that will allow the reader to quickly ascertain the nature of the technical disclosure. It is submitted with the understanding that it will not be used to interpret or limit the scope or meaning of the claims. In addition, in the foregoing Detailed Description, various features may be grouped together to streamline the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus the following claims are hereby incorporated into the Detailed Description, with each claim standing on its own as a separate embodiment.

What is claimed is:

1. A method for monitoring fluid in a region of a subject, the method comprising:
injecting an electrical first signal between a first and a second internal electrode, including injecting a portion of the first signal through at least a portion of the region;
measuring a resulting electrical second signal between a first and a second external electrode, electrically coupled to a weight scale device, including measuring the second signal between a first portion of the subject and a second portion of the subject spaced apart from the first portion; and
calculating a third signal indicative of the fluid in the region using one or both of the first and second signals.

2. The method of claim 1, wherein injecting the first signal between the first and second internal electrodes includes injecting the first signal through a thoracic region.

3. The method of claim 1, wherein injecting the first signal includes applying a voltage between the first and second internal electrodes using, at least in part, an implantable medical device.

4. The method of claim 3, wherein measuring the second signal includes measuring a voltage resulting from the applied voltage; and
wherein calculating the third signal includes using information about the resulting voltage.

5. The method of claim 1, wherein injecting the first signal includes injecting an electrical current between the first and second internal electrodes.

6. The method of claim 5, wherein measuring the second signal includes measuring a voltage resulting from the injected current; and
wherein calculating the third signal includes taking the ratio of the resulting voltage and the injected current.

7. The method of claim 5, wherein measuring the second signal includes measuring a voltage resulting from the injected current; and
wherein calculating the third signal includes calculating a product of the resulting voltage and the injected current.

8. The method of claim 1, further comprising determining a weight of the subject using the weight scale device, and
wherein calculating the third signal includes using information about the subject's weight in addition to information about the first and second signals.

9. The method of claim 1, wherein injecting the first signal includes injecting the first signal between an electrode disposed on a lead intermediate or distal end portion and a housing of an implantable medical device.

10. The method of claim 1, wherein injecting the first signal includes injecting the first signal between an electrode within, over, or about a left ventricle of a heart and an electrode near an upper portion of a left lung.

11. The method of claim 1, wherein injecting the first signal includes injecting the first signal between an electrode within, over, or about a left ventricle of a heart and an electrode within, over, or about a right ventricle of a heart.

12. The method of claim 1, further comprising detecting a change in an amount of fluid in the region.

13. The method of claim 12, further comprising alerting the subject or a caregiver in response to a specified detected change in the amount of fluid.

14. The method of claim 12, further comprising adjusting or initiating a therapy to the subject in response to a specified detected change in the amount of fluid.

15. An external weight scale device for use in a fluid monitoring system, the weight scale device comprising:
an external first electrode adapted to contact a subject's upper body portion;
an external second electrode adapted to contact a subject's lower body portion; and
a signal transmitter adapted to transmit a signal sensed by the first and the second electrodes to one or both of a remote processing unit or an electronic medical data storage.

16. The weight scale device of claim 15, wherein the first electrode comprises a handle electrode.

17. The weight scale device of claim 15, wherein the second electrode comprises a foot electrode.

18. The weight scale device of claim 17, further comprising at least an external third electrode, the third electrode and the foot electrode disposed on a top portion of the weight scale, the third electrode spaced apart from the foot electrode; and
wherein the signal transmitter is adapted to transmit a signal sensed by the foot and third electrodes to one or both of the remote processing unit or the electronic medical data storage.

19. The weight scale device of claim 15, further comprising a weight sensor; and
wherein information about the subject's weight is transmitted from the weight scale device to one or both of the remote processing unit or the electronic medical data storage via the signal transmitter.

20. The weight scale device of claim 15, wherein the signal sensed by the first and second electrodes comprises a voltage signal resulting from a signal generated by an implantable medical device and internally injected into the subject.

21. The weight scale device of claim 15, wherein the signal transmitter comprises one or both of a telemetry circuit or an antenna.

22. A device for measuring an indication of fluid in a region of a subject, the device comprising:
a receiver adapted to receive information about an electrical first and an electrical second signal, the first signal injected between a first and a second internal electrode positioned such that a portion of the first signal flows through a portion of the region, the second signal resulting between a first and a second external electrode electrically coupled to an external weight scale device;
a processing unit adapted to measure the indication of fluid in the region by calculating a third signal using one or both of the first and second signals; and
wherein one or both of the first and second signals are transmitted to the receiver via telemetry.

23. The device of claim 22, wherein the received information about the first signal comprises a value of an electrical current injected between the first and second internal electrodes.

24. The device of claim 22, wherein the received information about the first signal comprises a value of a voltage applied between the first and the second internal electrodes.

25. The device of claim 24, wherein the received information about the first electrical signal further comprises a measured value of a lead impedance; and
wherein the processing unit is adapted to calculate a value of an electrical current injected between the first and the second internal electrodes by dividing the applied voltage value by the lead impedance value.

26. The device of claim 22, wherein the received information about the second signal comprises a value of a voltage signal resulting from the injection of the first signal.

27. The device of claim 22, wherein the processing unit is adapted to detect a change in fluid in the region by detecting a change in a value of the third signal.

28. An external weight scale including the device of claim 22.

29. A machine usable medium including performable instruction for monitoring an amount of fluid in a region of a subject, comprising:
instructions that deliver a command to an implantable medical device to inject an electrical first signal between a first and a second internal electrode;
instructions that deliver a command to a weight scale to measure a resulting electrical second signal between a first and a second external electrode; and
instructions that calculate a third signal indicative of the amount of fluid in the region using one or both of the first and second signals.

30. The medium of claim 29, further comprising instructions that receive or store one or a combination of the first, second, or third signals.

31. The medium of claim 29, further comprising instructions that transmit one or a combination of the first, second, or third signals to an electronic medical data storage.

32. The medium of claim 29, further comprising instructions that determine an average value of two or more calculated third signals to which a subsequent third signal calculation may be compared thereby detecting a change in the amount of fluid.

33. The medium of claim 32, further comprising instructions that alert the subject or a caregiver to a specified change in the amount of fluid.

34. The medium of claim 33, further comprising instructions that deliver a command to the implantable medical device to adjust or initiate a therapy to the subject in response to the specified change in the amount of fluid.
